(19) United States
(12) Patent Application Publication
(10) Pub. No.: US 2022/0088044 A1
(43) Pub. Date: Mar. 24, 2022

(54) SUBSTANCES FOR TREATMENT OF CORONAVIRUS INFECTION

(71) Applicant: ScandiBio Therapeutics AB, Johanneshov (SE)

(72) Inventors: Adil Mardinoglu, Goteborg (SE); Jan Borén, Goteborg (SE); Mathias Uhlén, Lidingo (SE)

(21) Appl. No.: 17/025,396

(22) Filed: Sep. 18, 2020

(51) Int. Cl. A61K 31/706; A61K 31/198; A61K 31/4706; A61K 31/14; A61K 9/00; A61P 31/14

(52) U.S. Cl. CPC A61K 31/706; A61K 31/198; A61P 31/14; A61K 31/14; A61K 9/0053; A61K 31/4706

(57) ABSTRACT

There is provided a method of treating a human subject that is or has been infected with a coronavirus, comprising administration to the subject of:
A) serine, glycine, betaine, N-acetylglycine, N-acetylserine, dimethylglycine, sarcosine and/or phosphoserine;
B) N-acetyl cysteine, cysteine and/or cystine;
C) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine; and
D) nicotinamide riboside, quinolinate, deamino-NAD+, nicotinate D-ribonucleotide, nicotinamide D-ribonucleotide, nicotinate D-ribonucleoside, nicotinamide and/or nicotinate.

### TECHNICAL FIELD

The present disclosure relates to the treatment of a subject that is or has been infected with a coronavirus.

### BACKGROUND

Coronaviruses are a large family of ribonucleic acid viruses that typically cause mild to moderate upper respiratory diseases in humans. Members of this family are known to infect animals, including dogs, chickens, cattle, pigs, cats, pangolins, and bats. As far as we know, seven members of this family can infect humans and cause illness. Of these, HCoV-229E, -OC43, -NL63 and -HKU1 species cause mild respiratory diseases in humans. However, severe acute respiratory syndrome (SARS) that occurred in late 2002; Middle East Respiratory Syndrome (MERS-CoV) that emerged in 2012; and SARS-CoV-2, which appeared in China in December 2019, are the species that may cause serious respiratory diseases in humans. The epidemic of COVID-19 spread globally in a short time that classified as a pandemic by the World Health Organization (WHO). Since the beginning of the outbreak, infections have expanded rapidly into multiple simultaneous epidemics worldwide.

The phylogeny, virology, and epidemiology of SARS-CoV-2 is being studied extensively. At the genome level, SARS-CoV-2 has 79.5% homology to SARS□CoV-1, the causative agent of SARS in south China; 85% to 96% identity with bat□SL□CoVZC45, a bat SARS□like coronavirus, and 91.02% similarity with SARS-CoV-2-like coronavirus in pangolin named Pangolin-CoV. A population genetic analysis of 103 SARS-CoV-2 genomes from China revealed that causative agent of COVID-19 consists of two evolution types. Type L (70% prevalence in Wuhan), which is derived from the ancestral type S (30% prevalence in Wuhan), is more aggressive and contagious. Zhou et al. investigated the COVID-19 agent through full genome sequencing and phylogenetic analysis, classifying it as a betacoronavirus (a positive-sense, single-stranded RNA virus), the same subgenus as SARS-CoV as well as several bat coronaviruses. In addition, they proved that SARS-CoV-2 uses the same receptor binding (angiotensin-converting enzyme 2 (ACE2)) and cell entry pathway as SARS-CoV-1. In comparison with SARS-CoV-1, MERS-CoV is less closely related to SARS-CoV-2.

Epidemiologists in Wuhan believe the Huanan Seafood Wholesale Market in Wuhan to be the point of origin of SARS-CoV-2, due to its connection to the trading of live wild animals (WHO Jan. 22, 2020). COVID-19 is highly contagious, with a basic reproduction number (Ro) of between 1.4 and 6.5, and can be easily spread through coughs and sneezes, talking to infected persons, and touching the eyes, nose, or mouth after touching a contaminated surface.

SARS-CoV-2 has been found in stool and blood samples, but the rate of transmission, period of infectivity, and duration of viral shedding are uncertain and show great variation. Large-scale serologic screening, which is under development, may provide a better picture of statistics related to asymptomatic infections for epidemiologic analysis.

Signs of infection include respiratory symptoms, fever, cough, shortness of breath, and difficulty of breathing. In more serious cases, the infection can cause pneumonia, severe acute respiratory syndrome, kidney failure, and even death. As a result of the increase in deaths due to COVID-19, diagnosis with the real-time reverse transcription method (RT-PCR) was started relatively quickly, but due to the absence of an effective drug against this virus, patients are mainly kept under control with standard care treatment. Although the mortality rate is not known clearly, 11-14% case-death risk was reported in the first studies of severe patients and there has been reports of a case-death rate of approximately 2%. Further, many cases have resulted in hospitalization with complementary oxygen and sometimes pneumonia requiring more advanced ventilator support.

Currently, over 40 antiviral agents are accessible for the treatment of viral infections. Unfortunately, the most widely used antiviral drugs are accompanied by severe obstacles including insufficient selectivity, resistance, promotion of latency, toxicity or experimental difficulties. Drug repurposing approaches related to infectious diseases incorporate a variety of applications through the harmonizing key bioinformatics and cheminformatics methods to discover a drug target that could be repurposed in the combat against a viral pathogen. In addition to indisputable cost-effective benefits in the practice of drug discovery, recycled drugs gain access into clinical trials instantly or are taken up with compassionate use programs, particularly in the context of untreatable viral diseases or pandemics. Furthermore, drug repurposing provides an alternative source of data for a novel understanding of metabolic reprogramming in viral infections, as well as the organic compounds with previously unrecognized antiviral behaviours that are possible to expand in unveiling properties of virus biology. In various instances, drug repurposing identifies formerly undiscovered biomolecular networks, transforming them into novel pharmaceutical targets, even though the determined molecules may not be implemented into clinical trials.

### SUMMARY

### Introduction

In a previous study, the present inventors have found that the level of GSH is not enough to maintain and regulate the thiol redox status of the liver in subjects with high hepatic steatosis at fasting stage due to the depletion of glycine. Glycine can be synthesized via the interconversion of serine. It has been shown that the serine synthesis is downregulated in patients with NAFLD and supplementation of serine has attenuated alcoholic fatty liver by enhancing homocysteine metabolism in mice and rats. Depleted liver GSH is also restored by the administration of N-acetylcysteine as in acetaminophen poising. L-carnitine and nicotinamide riboside that both stimulate the transfer of fatty acids from cytosol to mitochondria have been identified as two additional cofactors that are depleted in patients.

To test the model-based predictions, we assessed the effect of short-term dietary supplementation with serine on fatty liver and fasting levels of plasma markers of liver functions in six obese subjects (BMI 32.5±2.70 kg/m2) with hepatic steatosis. Each patient received one oral dose of ˜20 g of L-serine (200 mg/kg) per day for 14 days. The supplementation was well-tolerated by all subjects. Results showed that ALT, AST, and ALP levels were significantly decreased after supplementation, and that the liver fat decreased significantly from 26.8±6.0% to 20.4±7.0%. We examined the concept by testing if supplementation of GSH and NAD+ precursors for two weeks would decrease high fat diet induced liver fat accumulation in mice. Therefore, we gave serine 300 mg/kg/day and nicotinamide riboside (NR) 400 mg/kg/day la gavage as well as 1 g/l of NAC (N-acetyl-L-cysteine) in the drinking water for 14 days to high fat diet-fed mice. Analysis of the liver lipids demonstrated a 50% reduction in hepatic triglycerides and a tendency to decreased levels of cholesterol esters. We also analysed the chain lengths of the hepatic triglycerides and found that shorter chain lengths of triglycerides (that are preferentially oxidized in the mitochondria) were significantly decreased after the supplementation. Hence, we demonstrated that supplementation of GSH and NAD+ precursors (i.e., the metabolites that were predicted to promote oxidation of fat in the liver), prevented hepatic steatosis in high fat diet-fed mice.

Pharmacological Data on Substances for Supplementation

N-acetylcysteine (NAC), also known as acetylcysteine, is the N-acetyl derivative of the amino acid L-cysteine and a precursor in the formation of the antioxidant glutathione in the body. Hence, administration of acetylcysteine replenishes glutathione stores.

Glutathione may act as endogenous neuromodulator, may also modulate the redox state of the NMDA receptor complex, activate ionotropic receptors that are different from any other excitatory amino acid receptor, and which may constitute glutathione receptors, potentially making it a neurotransmitter. As such, since N-acetylcysteine is a prodrug of glutathione, it may modulate all of the aforementioned receptors as well. It confers antioxidant effects and is able to reduce free radicals. Acetyleysteine also possesses some anti-inflammatory effects possibly via inhibiting NF-κB and modulating cytokine synthesis.

Indications:

NAC has two approved indications: (1) treatment of paracetamol (acetaminophen) overdose associated liver damage, and (2) to loosen thick mucus in individuals with cystic fibrosis or chronic obstructive pulmonary disease.

NAC has also been tested for contrast induced nephropathy, infertility, cystic fibrosis, ischemic heart diseases, HIV, hypercholesterolemia and schizophrenia. It is probably beneficial in any kind of acute hepatic failure. NAC usage in liver disease is discussed below.

Various study results indicating the role of NAC in lung injury and viral infectious diseases:
NAC is a thiol compound that acts directly as a free radical scavenger and as a precursor to reduced glutathione (GSH). This molecule has been shown to reduce the number and effect of COPD exacerbations and also the inflammatory response in epithelial cells exposed to the H₅N1 influenza A virus.
NAC improves inflammatory response and oxidative stress in patients with community-acquired pneumonia compared to conventional therapy.
In the presence of GSH or NAC (both strong radical eliminators), HDX (aspartic acid-β-hydroxamate, an inhibitor of endogenous SO₂ generating enzyme) failed to promote alveolar epithelial cell radical generation, PARP upregulation, caspase-3 activation or apoptosis, suggesting that the downregulated SO₂ pathway markedly facilitated the oxidative stress and thus likely induced apoptosis.
In the meta-analysis of 5 acute respiratory distress syndrome (ARDS) clinical trials, NAC did not contribute to reducing short-term mortality or 30-day mortality. However, the duration of stay in intensive care in these patients in the NAC group has been shortened.
In a randomized, placebo-controlled pilot study in HIV patients, 9 patients received NAC 900 mg twice daily, 8 patients received 1800 mg twice daily, and 7 patients received placebo. In this study, it was found that decreased glutathione levels in red blood cells increased significantly, red blood cell oxidized glutathione levels decreased significantly, and thus, NAC given at both doses compared to placebo led to generally non-significant increased glutathione: glutathione disulfide (GSH:GSSG) ratios. This suggests that NAC preparations may increase the ability of cells to neutralize higher levels of reactive oxygen species.

Pharmacological Properties:

NAC is soluble in water and alcohol, and practically insoluble in chloroform and ether. It is a white to white with light yellow cast powder and has a pKa of 9.5 at 30° C. NAC is stable in gastric and intestinal fluids and rapidly absorbed after oral administration. It is not affected by food intake. It reaches peak plasma concentration in 30-60 minutes after application. The distribution volume (Vd) is between 0.33 and 0.47 L/kg, which is evident in extracellular fluids and passes primarily to the lung, kidney, and liver. After oral administration, 48% of the amount passed to the blood is determined in the lungs. The rate of binding to plasma proteins is about 50%. NAC is extensively liver metabolized, and 22-30% is excreted in urine in the form of sulfate and taurine. NAC has a half-life of 5.6-6 hours in adults.

Dosage:

NAC is available as intravenous and oral formulations. The IV injection and inhalation preparations are, in general, prescription only, whereas the oral solution and the effervescent tablets are available over the counter in many countries including the United States. Daily dose of NAC is 200 mg capsules taken 3 times a day (morning, lunch, evening) or a single dose of 600 mg (3 capsules) in the evening. In paracetamol poisoning the loading dose is ˜140 mg/kg, and the maintenance dose is 70 mg/kg every 4 hours (17 doses).

In other indications, NAC has been tested in various doses. In early psychosis, NAC supplementation has been given at a dose of 2700 mg/day for 6 months in a double-blind randomized controlled trial. NAC supplementation is used to enhance performance in elite sport. A recent review of the literature evaluated the effect of NAC supplementation. The typical daily dose of NAC reported was 5.8 g/day; with a range between 1.2 and 20.0 g/day. In people exposed to asbestos, effect of NAC supplementation on oxidative stress status and alveolar inflammation was analyzed in a double-blind, randomized clinical trial using a dose of 1800 mg/day for 4 months. In HIV-infected patients, NAC was administered in oral doses of 6,000-8,000 mg daily for several months. It had a good safety profile, adverse effects were minimal and not significantly associated with NAC ingestion.

Side Effects:

Side effects occur rarely with NAC usage. Even at very high doses, serious adverse events or signs of intoxication are not observed. Most common adverse reactions (incidence greater than 2%) are rash, urticaria/facial flushing, pruritus, nausea and vomiting. Adverse effects for oral formulations of acetylcysteine have been reported to include nausea, vomiting, rash, and fever.

Although unclear, there was a trend of increasing side effects with increasing doses and IV usage of NAC compared with placebo. Anaphylactic reactions, i.e. pruritus, rash, angioedema, bronchospasm, tachycardia, hypotension, have been previously reported to occur within 30 min after IV loading dose of NAC in ˜3-6% of people. Hypersensitivity reactions, including generalized urticaria have been observed in patients receiving oral acetylcysteine for acetaminophen overdose, but anaphylaxis has rarely been reported with oral administration.

Occasionally severe and persistent vomiting occurs as a symptom of acute acetaminophen overdose. Treatment with NAC may aggravate the vomiting and increase the risk of upper gastrointestinal haemorrhage in at risk patients (e.g., those with oesophageal varices, peptic ulcers, etc.).

Large doses in a mouse model showed that acetylcysteine could potentially cause damage to the heart and lungs. It has been found that acetylcysteine was metabolized to S-nitroso-N-acetylcysteine (SNOAC), which increased blood pressure in the lungs and right ventricle of the heart (pulmonary artery hypertension) in mice treated with acetylcysteine. The effect was similar to that observed following a 3-week exposure to an oxygen-deprived environment (chronic hypoxia). It was also found that SNOAC induced a hypoxia-like response in the expression of several important genes both in vitro and in vivo.

Overdose and Treatment:

No specific antidote available. Supportive and symptomatic treatment are performed.

Contraindications:

NAC is contraindicated in patients with previous allergic/anaphylactoid reaction to acetylcysteine. Patients with acute asthma attacks can also not use NAC. Pregnancy risk category of NAC is B. There is insufficient data on the usage in pregnancy and lactation. Therefore, it should only be used if it is very necessary. Drug interaction with acetylcysteine is very rare. Minor interactions have been reported with parenteral nitro-glycerine and oral forms of nitrates. NAC has no effect on the use of vehicles and machinery.

L-carnitine (levocarnitine) is a naturally occurring substance required in mammalian energy metabolism. It is a carrier molecule that facilitates the transport of long-chain fatty acids across the inner mitochondrial membrane, thereby delivers substrate for oxidation and subsequent energy production.

Carnitine deficiency is characterized with very low L-carnitine levels in plasma and tissues and may be either primary or secondary. Primary carnitine deficiency is an autosomal recessive disorder caused by a deficiency in the plasma membrane carnitine transporter and leads to urinary carnitine wasting. SLC22A5 mutations can also affect carnitine transport and decrease carnitine levels. Secondary carnitine deficiency is associated with various causes like inadequate intake, decreased synthesis due to liver disorders, loss of carnitine during diarrhoea, diuresis or haemodialysis. In primary systemic deficiency, the clinical presentation consists of recurrent episodes of Reye-like encephalopathy, hypoketotic hypoglycaemia, and/or cardiomyopathy. Associated symptoms include hypotonia, muscle weakness and failure to thrive. In some patients, particularly those presenting with cardiomyopathy, carnitine supplementation may rapidly alleviate signs and symptoms.

Indications:

L-carnitine is indicated in the treatment of primary and secondary L-carnitine deficiency. It may also be used in patients taking certain drugs (such as valproic acid for seizures or antibiotics for tuberculosis), or during medical procedures (haemodialysis for kidney disease) that deplete the body's L-carnitine. (27)

L-carnitine has also been tested in heart failure, ischemic heart diseases, peripheral arterial diseases, HIV, male infertility, anorexia, chronic fatigue syndrome and fatigue associated with chronic diseases and chronic obstructive pulmonary disease. It is also used as a replacement supplement in strict vegetarians, dieters, and low-weight or premature infants. In athletes, carnitine has been used to improve performance, but beneficial effects in athletes is not consisted in all studies.

Various study results indicating the role of L-carnitine in lung injury and viral infectious diseases:

The results of a study using a lamb model with increased pulmonary blood flow showed that chronic L-carnitine treatment alleviates changes in lung carnitine homeostasis, reduces associated oxidative stress, and improves pulmonary mitochondrial function, NO signalling and eventually endothelial function. Chronic L-carnitine treatment may improve and/or decrease the reduction in endothelial function in children with congenital heart diseases, but more research is needed.

It has been reported that 20 mg/kg/day carnitine supplement to premature new-borns increases total carnitine concentrations in plasma and red blood cells, has positive effects on growth and can improve periodic breathing.

In addition to standard treatments for patients with chronic hepatitis C, the addition of 2 grams of Acetyl-L-Carnitine supplements 2 times a day has been shown to increase daily activity in patients, reduce fatigue and have a lower productivity reduction compared to the placebo group.

Pharmacological Properties:

Levocarnitine is a white crystalline, hygroscopic powder. It is readily soluble in water, hot alcohol, and insoluble in acetone.

The absolute bioavailability of levocarnitine is ˜15-16%. The mean distribution half-life is ˜0.6 hours and the mean apparent terminal elimination half-life is 17.4 hours. Total body clearance of levocarnitine (Dose/AUC including endogenous baseline concentrations) is a mean of 4.00 L/h. Levocarnitine is not bound to plasma protein or albumin when tested at any concentration.

Major metabolites are trimethylamine N-oxide, primarily in urine (8% to 49% of the administered dose) and [3H]-γ-butyrobetaine, primarily in faeces (0.44% to 45% of the administered dose). Urinary excretion of levocarnitine is about 4 to 8% of the dose. Faecal excretion of total carnitine is less than 1% of the administered dose.

Dosage:

A levocarnitine dosage of 1 to 3 g/day has been recommended for a 50 kg subject.

The following doses have been studied in scientific research. In patients with carnitine depletion in peripheral blood mononuclear cells, L-carnitine supplementation has been given at a dose of 6 g/day for 2 weeks. Several studies have tested if L-carnitine supplementation promotes weight loss in obese subjects (4 g/L, 8 weeks). Efficacy and effectiveness of carnitine supplementation for cancer-related fatigue has recently been tested in a systematic literature review and meta-analysis (nine studies used a dose between 2 to 6 g per day). Impact of L-carnitine supplementation on plasma lipoprotein (a) concentrations have been analysed in a recent systematic review and meta-analysis of randomized controlled trials (studies used 2-4 g/day).

A systematic review of databases (PubMed, EMBASE, and the Cochrane Library) has been conducted to determine the effects of L-carnitine on all-cause mortality and cardiovascular morbidities in the setting of acute myocardial infarction (meta-analysis of five controlled trials, n=3108). There were no significant differences between the effects of daily L-carnitine supplementation of 2 g and 6 g on heart failure, unstable angina, or myocardial reinfarction

Side Effects:

L-carnitine is usually well tolerated. Various mild gastrointestinal complaints have been reported during the long-term administration of oral L- or D-carnitine; these include transient nausea and vomiting, abdominal cramps, and diarrhoea. At doses of approximately 3 g/day, carnitine supplements can cause nausea, vomiting, abdominal cramps, diarrhoea, and a “fishy” body odor. Rarer side effects include muscle weakness/mild myasthenia in uremic patients and seizures in those with seizure disorders.

Gastrointestinal adverse reactions with levocarnitine may be avoided by a slow consumption of the solution or by a greater dilution. Decreasing the dosage often diminishes or eliminates drug-related patient body odor or gastrointestinal symptoms when present. Tolerance should be monitored very closely during the first week of administration, and after any dosage increases.

Some research indicates that intestinal bacteria metabolize carnitine to form a substance called TMAO that might increase the risk of cardiovascular disease. This effect appears to be more pronounced in people who consume meat than in vegans or vegetarians. The implications of these findings are not well understood and require more research.

Overdose and Treatment:

There have been no reports of toxicity from levocarnitine over dosage. Levocarnitine is easily removed from plasma by dialysis. Large doses of levocarnitine may cause diarrhoea.

Contraindications:

There are no known contraindications. Pregnancy risk category of L-carnitine is B. Because animal reproduction studies are not always predictive of human response, it should be used during pregnancy only if clearly needed. Drug interaction with L-carnitine is very rare. It may decrease the effectiveness of the thyroid hormone and increase the effects of warfarin.

Nicotinamide riboside (NR). Nicotinamide adenine dinucleotide (NAD) is an endogenous substance that is involved in several important cell functions such as signal transduction, DNA repair, and post-translational protein modifications. NAD consuming activities and cell division necessitate ongoing NAD synthesis, either through a de novo pathway that originates with tryptophan or via salvage pathways from three NAD+ precursor vitamins, nicotinamide riboside, nicotinamide, and nicotinic acid.

In animals, NAD generation is vital since it is linked to several redox reactions in the body. NAD plays a central role in energy metabolism and oxidative phosphorylation and is a key component of many metabolic pathways for carbohydrates, lipids, and amino acids.

Indications:

NR has been used as an NAD+ precursor vitamin. NR is considered a form of vitamin B3 (niacin) and it is available as an over-the-counter dietary supplement, and approved as food ingredient in enhanced water products, protein shakes, nutrition bars, gum and chews at no more than 0.027% by weight. Human blood NAD+ can rise as much as 2.7-fold with a single oral dose of NR in a pilot study, and single doses of 100, 300 and 1,000 mg of NR produce dose-dependent increases in the blood NAD+ metabolome in the first clinical trial of NR pharmacokinetics in humans.

Various study results indicating the role of niacin in lung injury and viral infectious diseases:
In a study in rats with endotoxemia, high doses of niacin have been shown to regulate the nuclear factor kappa B pathway, alleviate lung inflammation, reduce histological lung injuries, and increase survival during sepsis.
Niacin has been reported to alleviate LPS-induced acute lung injury, possibly by preventing the depletion of NAD.
In a randomized, double-blind, placebo-controlled study in HIV patients receiving antiretroviral therapy and with dyslipidemia with insulin resistance, patients were randomized into 5 groups. Group 1: group without any life change or supplement, Group 2: group with low-fat diet and exercise added, Group 3: group with low-fat diet and exercise, as well as fenofibrate; Group 4: Niacin in addition to low-fat diet and exercise, and Group 5: Group with fenofibrate and niacin in addition to low-fat diet and exercise. As a result of the study, in addition to low-fat diet and exercise, the addition of fenofibrate and niacin to the patients receiving antiretroviral therapy increased HDL cholesterol. It has been reported to be effective and safe to reduce hypertriglyceridemia and to improve hypoadiponectinemia.
It has been shown that providing patients with HIV infection with atherogenic dyslipidemia, extended-release niacin supplementation at doses up to 2000 mg daily is effective, safe, and well tolerated. Increases in glycemia and insulin resistance have been reported to be temporary in individuals in this study.
A randomized controlled study determined the short-term effects of extended-release niacin (ERN) on endothelial function, measured by flow-mediated vasodilation (FMD) of the brachial artery, in HIV-infected adults with low HDL-c. This pilot study demonstrated that short-term niacin therapy could improve endothelial function in HIV-infected patients with low HDL-c study.
In a study conducted in 2020, it was been shown that SARS-CoV-2 infection and PARP expression dysregulate the NAD Metabolome and SARS-CoV-2 induces a set of poly(ADP-ribose) polymerase (PARP) family members. PARP enzymes are enzymes that induce natural immunity against mouse hepatitis virus (MHV), which is used as a coronavirus infection model. MHV infection has been shown to increase NAD and NADP in natural immune cells. Study data show that overexpression of virally induced PARP and PARP10 depress host cell NAD metabolism and NAD+enhancement strategies differ in restoring PARP10. These data showed that, not nuclear NAD+, but the cytoplasmic NAD+ increase can regulate antiviral PARP functions that support the natural immune response to other viruses and coronaviruses susceptible to PARP-mediated antiviral activity.

Dosage and Toxicity:

A typical dose may be 100-250 mg taken before the first meal of the day. In a study, there was no mortality at an oral dose of 5000 mg/kg. Based on the results of a 14-day study, a 90-day study was performed comparing NR at 300, 1000, and 3000 mg/kg/day to an equimolar dose of nicotinamide at 1260 mg/kg/day as a positive control. Results from the study show that NR had a similar toxicity profile to nicotinamide at the highest dose tested. The lowest observed adverse effect level for NR was 1000 mg/kg/day, and the no observed adverse effect level was 300 mg/kg/day.

Airhart et al. (PLoS One. 2017 Dec. 6; 12(12)) recently reported an open-label, non-randomized study of the pharmacokinetics of NR and its effects on blood NAD+ levels. In eight healthy volunteers, 250 mg NR was orally administered on days 1 and 2, then up-titrated to peak dose of 1000 mg twice daily on days 7 and 8. On the morning of day 9, subjects completed a 24-hour pharmacokinetic study after receiving 1000 mg NR at t=0. Whole-blood levels of NR, clinical blood chemistry, and NAD+ levels were analysed. Results showed that oral NR was well tolerated with no adverse events. Significant increases comparing baseline to mean concentrations at steady state were observed for both NR (p=0.03) and NAD+ (p=0.001); the latter increased by 100%. Absolute changes from baseline to day 9 in NR and NAD+ levels correlated highly (R2=0.72, p=0.008). The authors concluded that because NR increases circulating NAD+ in humans, NR may have potential as a therapy in patients with mitochondrial dysfunction due to genetic and/or acquired diseases.

Side Effects:

In 2016, FDA granted NR GRAS (Generally Recognized as Safe) status on the basis of existing clinical study, which showed “no observed adverse effect level (NOAEL) was 300 mg/kg/day”. NAD seems safe for most people when used appropriately and short-term, up to 12 weeks.

Flushing is the most sensitive end point of nicotinic acid effects, but after ingestion of supplemental nicotinamide, no cases of flushing or glucose intolerance have been reported and only one case of hepatitis was reported following the ingestion of greater than 3 g/day for several days. Such side-effects have not been reported for NR.

NR was not defined as genotoxic but data on the use of NAD during pregnancy and breast-feeding is inadequate. European Food Safety Authority (EFSA) EFSA has issued a positive opinion of nicotinamide riboside chloride as a novel food on Aug. 7, 2019. And a dose of 230 mg/day in pregnant and lactating women is permissible (EFSA Opinion: https://www.efsa.europa.eu/en/efsajournal/pub/5775).

Interactions:

There is no known interaction with NAD usage.

L-serine is generally classified as a non-essential amino acid, however under certain circumstances, vertebrates cannot synthesize it in sufficient quantities to meet necessary cellular demands. L-serine is biosynthesized in the mammalian central nervous system from 3-phosphoglycerate and serves as a precursor for the synthesis of the amino acids glycine and cysteine.

Physiologically, it has a variety of roles, perhaps most importantly as a phosphorylation site in proteins. Mutations in the metabolic enzymes that synthesize L-serine have been implicated in various human diseases.

Indications:

Hereditary sensory and autonomic neuropathy type 1 (HSAN1) is a disorder caused by missense mutations in the enzyme serine palmitoyl transferase (SPT). Subjects received daily supplements of powdered L-serine (mixed in water) on a low- or high-dose schedule (200 or 400 mg/kg body weight, respectively, n=7 per group). Results showed that an altered substrate selectivity of the mutant SPT is key to the pathophysiology of HSAN1 and raise the prospect of L-serine supplementation as a first treatment option for this disorder.

Various study results indicating the role of serine in immune system diseases:
Pharmacological inhibition of de novo serine synthesis in vivo decreased LPS induction of IL-1β levels and improved survival in an LPS-driven model of sepsis in mice, which reveals that serine metabolism is necessary for GSH synthesis to support IL-1β cytokine production.
A study showed that extracellular serine is required for optimal T cell expansion even in glucose concentrations sufficient to support T cell activation, bioenergetics, and effector function. Restricting dietary serine impairs pathogen-driven expansion of T cells in vivo, without affecting overall immune cell homeostasis. Mechanistically, serine supplies glycine and one-carbon units for de novo nucleotide biosynthesis in proliferating T cells, and one-carbon units from formate can rescue T cells from serine deprivation.
L-serine has been shown to decrease the macrophage- and neutrophil-mediated inflammatory responses in mice during Pasteurella multocida infection.
L-serine administration has been reported to reduce body weight by decreasing orexigenic peptide expression and reduces oxidative stress and inflammation during aging in mice, possibly by modulating the Sirt1/NFκB pathway.
Reduction of inflammatory responses (caspase-3, TNF-α, IL-1β and IL-6 and the number of GFAP- and Iba-1-positive cells) by L-serine treatment has been shown to lead to neuroprotection in mice after traumatic brain injury.

Safety:

In a recent study, Fridman et al (Neurology. 2019; 92(4)) performed a randomized, double-blind, placebo-controlled trial (n=18) to evaluate the efficacy and safety of L-serine treatment for adults with hereditary sensory and autonomic neuropathy type 1 (HSAN1). The study subjects were randomized to L-serine (400 mg/kg/day) or placebo for one year. All participants received L-serine during the second year. Analysis of vital signs, physical examination findings, and clinical laboratory examinations, did not reveal adverse effects of L-serine. Thus, long-term L-serine supplementation did not reveal adverse effects of L-serine.

FDA determined that L-serine is generally safe (GRAS), and it also appears to be neuroprotective.

Serine supplementation (three daily doses of 5 g of L-serine [i.e., 190 mg/kg]) has also been shown to be safe even in pregnancy, as shown by in pre- and postnatal treatment of 3-phosphoglycerate-dehydrogenase deficiency.

Rationale of the Composition and Dosages

The present inventors have recently integrated kinetic data from human turn-over studies using stable-isotope technique with experimentally derived flux data to simulate the dynamics of liver metabolism of subjects with varying degree of hepatic steatosis using personalized liver genome-scale metabolic models (GEMs). The correlations between the predicted intracellular fluxes of the liver and hepatic steatosis were assessed to identify underlying molecular mechanisms that are disturbed in subjects with NAFLD. The systems level analysis indicated that altered NAD+ and GSH metabolism (with increased demand for NAD+ and GSH in NAFLD) was a prevailing feature in NAFLD.

Next, the present inventors modelled and validated if it would be possible to reverse hepatic steatosis by boosting the NAD+ and GSH metabolism (as described above). Advanced modelling was performed to clarify the molar relationship of the co-factors needed for optimal efficiency. It was found that the metabolites serine, L-carnitine, NAC and NR in a molar ratio of 30:4:4:1 may effectively promote NAD+ and GSH metabolism and decrease the amount of hepatic steatosis. Hence, suitable daily supplementation doses are 24.7 g serine (0.0078×30 moles), 5.1 g of L-carnitine (given in the stable salt form of 7.46 g L-carnitine tartrate) (0.0078×4 moles), 5.1 g of NAC (0.0078×4 moles) and 2 g of NR (0.0078 moles). This is further discussed below.

In a 7-day rat toxicology study, we assessed the tolerability of these four metabolites/supplements at intended clinical doses (Formulation I), and at 10-fold (Formulation II) and 30-fold (Formulation III) dose levels. Several clinical observations (e.g. ploughing with the nose in the bedding material and excessive chewing) were made in all groups in connection with, or shortly after dosing, and are not considered of toxicological significance. Most severe clinical observations such as ataxia, cyanosis, irregular respiration and/or decreased motor activity were observed in the group given Formulation III at Days 1 and 2. This led to a lowering of the dose (33%) in this group on Day 3. The new dose level was well tolerated for the remainder of the study. In all groups, some milder signs of discomfort (eyes half-shut and pilo-erection) were observed at Day 1 but were not present from Day 2 in Groups 1 and 2. This finding indicates a reaction to a new, unknown treatment, but may also indicate a tolerability build-up after repeated exposure. At the end of 7 days, none of the doses caused significant alterations in haematological and plasma chemistry parameters, and also in clinical pathology analysis or during the macroscopic analysis at necropsy.

Second, 5-day human calibration study was performed by supplementing 20 g/day L-serine, 3 g/day L-carnitine, 5 g/day NAC and 1 g/day NR and measuring the kinetics of these supplements/metabolites in the plasma of nine subjects. Both on targeted and untargeted metabolomics, the plasma levels of the cofactors were quantified, and it was showed that such supplementation increased the plasma levels of each metabolite proportionally. No significant change was detected in the plasma levels of glucose, insulin, free fatty acids, triglycerides, total cholesterol, HDL, LDL and known liver markers including gamma GT, bilirubin, ALP, AST and ALT. The plasma levels of the key metabolites associated with liver fat and insulin resistance showed significant decreases, but plasma level of glucose remained stable even though participants did not have any food or drink during the 9-hours fasting calibration study. Next, untargeted metabolomics data were generated to reveal the changes associated with the supplementation of these metabolic cofactors using genome-scale metabolic modelling and observed that such supplementation is significantly associated with lipid, amino acid and antioxidant metabolism.

Finally, an ordinary differential equation (ODE) model were generated to predict blood concentrations during daily long-term supplementation of the four supplements. Liver concentrations were predicted using pharmacokinetic modelling to adjust the doses of the individual supplements in human clinical studies. Using the targeted metabolomics data obtained from the plasma of nine subjects, a three-compartment ODE pharmacokinetic model was developed to represent metabolic cofactors distribution within the body. The model structure was inferred from a mechanistic view of holdup in the stomach and absorption from the small intestine to the blood, with central clearance. Interpolations of the plasma concentrations of each supplement were constructed for every subject. The mean of the interpolations was used as the target concentration curve and the model was subsequently fitted to this curve.

Model fit to the mean plasma serine level predicted that a twice-daily dose of 12.4 g serine might produce a desired long-term increase in mean plasma serine concentration of 100%. Doses up to 400 mg/kg/day (around 25-30 g/day) have been studied in humans and shown to be safe.

For L-carnitine, the model predicted that a twice-daily dose of 8.2 grams would produce a desired long-term increase in mean plasma concentration of 100%. However, since long term supplementation studies for the safety of L-carnitine above 7 grams per day have not been examined, the dose was reduced to 2.55 grams twice-daily. This resulted in a long-term increase in mean plasma concentration of 31%, which was considered a reasonable trade-off between risk of toxicity and increase in L-carnitine plasma concentration.

Interpolations of NAC and model fit to the mean plasma NAC level predicted that a twice-daily dose of 2.47 grams NAC would produce a desired long-term increase in mean plasma NAC concentration of 100%. A daily dosage of 4-6 grams of NAC have been shown to be safe in humans.

Regarding the dose adjustment of the NR, we used existing literature studies rather than performing additional modelling. Considering the published experimental data, we decided to supplement 1 g of NR twice daily.

Based on the results of the ODE modelling, it was concluded that supplementation of 12.35 g serine, 2.55 g of L-carnitine, 2.55 g of NAC and 1 g of NR twice-daily can be used for effective treatment of subjects. Considering that L-carnitine tartrate (salt form of L-carnitine) contains 67.6% of L-carnitine, 3.75 g L-carnitine tartrate twice-daily can be used for effective treatment of subjects.

Considering metabolic pathways, the present inventors have identified the following alternatives to the above-mentioned substances:

| Substance | Alternatives |
|---|---|
| serine | glycine, betaine, N-acetylglycine, N-acetylserine, |
| | dimethylglycine, sarcosine and/or phosphoserine |
| NAC | Cysteine and/or cystine |
| L-carnitine | deoxycarnitine, gamma-butyrobetaine, 4- |
| | trimethylammoniobutanal, 3-hydroxy-N6,N6,N6- |
| | trimethyl-L-lysine, N6,N6,N6-trimethyl-L-lysine |
| | and/or lysine |
| NR | quinolinate, deamino-NAD+, nicotinate D- |
| | ribonucleotide, nicotinamide D-ribonucleotide, |
| | nicotinate D-ribonucleoside, nicotinamide and/or |
| | nicotinate |

Based on the discussion above and other scientific insights, the present inventors hypothesized that a combination of the four substances could be used for treating patients that are or have been infected with SARS-CoV-2. As shown the Example section below, the hypothesis was confirmed.

To obtain the therapeutic effect, it is not necessary to always include all four substances. The inventors have however identified serine (or one or more of its alternatives) as the most important substance and NR (or one or more of its alternatives) as the second most important substance. Further, the inventors have found that the optimal daily molar dose is higher for serine than for NR.

Accordingly, the present disclosure provides a method of treating a subject that is or has been infected with a coronavirus, comprising administration to the subject of:

A) serine, glycine, betaine, N-acetylglycine, N-acetylserine, dimethylglycine, sarcosine and/or phosphoserine;
B) N-acetyl cysteine, cysteine and/or cystine;
C) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine; and
D) nicotinamide riboside, quinolinate, deamino-NAD+, nicotinate D-ribonucleotide, nicotinamide D-ribonucleotide, nicotinate D-ribonucleoside, nicotinamide and/or nicotinate.

There is also provided a method of treating a subject suffering from a SARS, comprising administration to the subject of:

A) serine, glycine, betaine, N-acetylglycine, N-acetylserine, dimethylglycine, sarcosine and/or phosphoserine;
B) N-acetyl cysteine, cysteine and/or cystine;
C) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine; and
D) nicotinamide riboside, quinolinate, deamino-NAD+, nicotinate D-ribonucleotide, nicotinamide D-ribonucleotide, nicotinate D-ribonucleoside, nicotinamide and/or nicotinate.

In an embodiment, the SARS is COVID-19.

### BRIEF DESCRIPTION OF THE DRAWINGS

FIG. 1 shows a Kaplan-Meier graph of the proportion of patients in the treated group and the placebo group that had symptoms on each day.

### DETAILED DESCRIPTION

As a first aspect of the present disclosure, there is provided a composition for use in a therapeutic method. The therapeutic method is:

a method of treatment of a subject that is or has been infected with a coronavirus, typically a severe acute respiratory syndrome-related coronavirus; or
a method of treatment of a subject suffering from a severe acute respiratory syndrome (SARS).

SARS is a viral respiratory disease of zoonotic origin caused by a severe acute respiratory syndrome coronavirus. In an embodiment, the SARS is COVID-19.

In an embodiment, the severe acute respiratory syndrome-related coronavirus is severe acute respiratory syndrome coronavirus 2 (SARS-CoV-2).

The composition of the first aspect comprises:

A) serine, glycine, betaine, N-acetylglycine, N-acetylserine, dimethylglycine, sarcosine and/or phosphoserine;
B) N-acetyl cysteine, cysteine and/or cystine;
C) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine; and
D) nicotinamide riboside, quinolinate, deamino-NAD+, nicotinate D-ribonucleotide, nicotinamide D-ribonucleotide, nicotinate D-ribonucleoside, nicotinamide and/or nicotinate.

In a variant of the first aspect, the composition comprises A), B), C) and optionally D).

In group A), serine and glycine are preferred. The most preferred substance in group A) is serine, which is typically provided as L-serine.

In group B), N-acetyl cysteine (NAC) and cysteine are preferred. The most preferred substance in group B) is NAC.

The substance of group C) is preferably carnitine, optionally in the form of a carnitine salt, such as carnitine tartrate. Most preferably, the substance of group C) is L-carnitine, optionally in the form of a L-carnitine salt, such as L-carnitine tartrate.

The substance of group D) is preferably nicotinamide riboside (NR).

The substance(s) of group A) is preferably included in a higher molar amount than the substance(s) of group D). When efficacy and toxicity is also considered, the molar ratio of A) to D) is normally between 250:1 and 1.5:1 and typically between 150:1 and 3:1. Preferably, the molar ration is between 90:1 and 10:1, more preferably between 50:1 and 20:1.

The molar ratio of A) to B), considering efficacy and toxicity, is typically between 20:1 and 1:4, such as between 16:1 and 1:4, preferably between 12:1 and 1.5:1 and more preferably between 10:1 and 3:1.

In embodiments including the substance(s) of group C), the molar ratio of A) to C), considering efficacy and toxicity, is normally between 150:1 and 1:1, typically between 100:1 and 2:1, preferably between 30:1 and 3:1, more preferably between 15:1 and 4:1.

The above ratios entail that a patient consuming the composition can obtain appropriate doses of the respective substances.

In one embodiment, the composition of the first aspect is a solid, such as a solid powder. Such a powder can be mixed with water, e.g. by the patient/consumer, a nurse or a physician. In another embodiment, the composition of the first aspect is an aqueous solution or suspension (“cocktail”), which facilitates convenient oral administration. Such an aqueous solution or suspension is preferably ready to drink.

As a particularly preferred embodiment of the first aspect, the composition is a powder comprising:

A) serine;
B) N-acetyl cysteine;
C) carnitine; and
D) nicotinamide riboside, wherein
the molar ratio of A) to B) is between 16:1 and 1.5:1, preferably between 10:1 and 3:1, the molar ratio of A) to C) is between 100:1 and 2:1, preferably between 30:1 and 3:1, more preferably between 15:1 and 4:1; and the molar ratio of A) to D) is between 150:1 and 3:1, preferably between 90:1 and 10:1, more preferably between 50:1 and 20:1.

As another particularly preferred embodiment of the first aspect, the composition is a powder comprising:

A) serine;
B) N-acetyl cysteine and/or cysteine;
C) optionally carnitine; and
D) nicotinamide riboside, wherein
the molar ratio of A) to D) is between 90:1 and 10:1, preferably between 50:1 and 20:1, more preferably between 40:1 and 20:1.

In embodiments of the solution or suspension according to the first aspect:
the concentration of A) is typically 0.20-2.4 mmol/ml, preferably 0.40-2.4 mmol/ml and more preferably 0.60-2.4 mmol/ml;
the concentration of B) is normally 0.09-0.90 mmol/ml, typically 0.09-0.54 mmol/ml, preferably 0.11-0.40 mmol/ml and more preferably 0.013-0.30 mmol/ml; and/or
the concentration of D) is typically 0.006-0.12 mmol/ml, preferably 0.012-0.08 mmol/ml and more preferably 0.018-0.07 mmol/ml.

When included in the solution or suspension according to the first aspect:
the concentration of C) is normally 0.009-0.38 mmol/ml, typically 0.009-0.19 mmol/ml, preferably 0.016-0.16 mmol/ml and more preferably 0.028-0.12 mmol/ml.

The solution or suspension of the first aspect may be provided in a package for convenient handling and distribution. Further, the volume of such a package may be such that drinking the whole contents of the package at once or during a single day results in oral administration of appropriate doses of the substances in the solution or suspension. In one embodiment, the volume of the package is 25-1000 ml. The volume is preferably 50-500 ml. When it is intended that the consumer/patient shall drink more than one package per day, the volume is typically relatively low, such as 25-500 ml, preferably 25-400 ml.

In one embodiment, the packaged solution or suspension comprises 48-478 mmol of A). Thereby, the dose of A) is effective, but not toxic. In a preferred embodiment, A) is serine in an amount of 5-50 g, more preferably 10-50 g.

In an alternative of complimentary embodiment, the packaged solution or suspension comprises 2.0-39.2 mmol of D) when D) is NR and 2.0-196 mmol of D) when D) is not NR. Thereby, the dose of D) is effective, but not toxic. In a preferred embodiment, D) is NR in an amount of 0.5-10 g, more preferably 1.5-6 g.

When the composition of the first aspect is a powder, it may also be packaged. As an example, the powder may be provided in unit dose packs. It follows from the discussion above that such a unit dose may comprise 48-478 mmol of A) and/or 2.0-39.2 mmol of D) when D) is NR and 2.0-196 mmol of D) when D) is not NR. Further, such as packed powder preferably comprises serine in an amount of 5-50 g and/or NR in an amount of 0.5-10 g. More preferably, such a packed powder comprises serine in an amount of 10-50 g and/or NR in an amount of 1.5-6.0 g.

The substances of the present disclosure are preferably a significant part of the composition of the first aspect. For example, the substances included in groups A)-D) may amount to at least 10%, such as at least 25%, such as at least 50% of the dry weight of the composition of the first aspect. In one embodiment, the weight of serine is at least 10%, such as at least 25%, such as at least 40% of the dry weight of the composition of the first aspect.

The composition of the first aspect may comprise one or more tasting agent(s), such as one or more sweetener(s) (e.g. sucralose) and/or one or more flavor agent(s). It may also comprise a lubricant, such as a polyethylene glycol lubricant (e.g. Polyglykol 8000 PF (Clariant)).

In an embodiment, the therapeutic method comprises oral administration of the composition.

The therapeutic method of the first aspect may further comprise administration of an anti-viral drug.

In one embodiment, the therapeutic method of the first aspect further comprises administration of chloroquine or hydroxychloroquine. In case of hydroxychloroquine, the daily dose may be in the range of 100-1200 mg, such as 200-1000 mg. As an example, hydroxychloroquine may be given for a period of 2-8 days, such as 3-7 days.

The therapeutic method may for example comprise administration, such as oral administration, of:

A) in a dose of 0.48-24 mmol/kg/day, such as 0.48-4.8 mmol/kg/day, such as 1.8-4.8 mmol/kg/day, such as 2.9-4.7 mmol/kg/day;
B) in a dose of 0.31-3.05 mmol/kg/day, such as 0.31-1.84 mmol/kg/day, such as 0.40-1.23 mmol/kg/day;
optionally C) in a dose of 0.100-2.50 mmol/kg/day, such as 0.200-2.00 mmol/kg/day, such as 0.230-1.00 mmol/kg/day, such as 0.300-0.800 mmol/kg/day; and/or
D) in a dose of 0.020-1.96 mmol/kg/day, such as 0.020-0.39 mmol/kg/day, such as 0.039-0.31 mmol/kg/day, such as 0.059-0.24 mmol/kg/day, provided that the dose is not higher than 0.39 mmol/kg/day when D) is NR.

If the doses are not adjusted to the weight of the patient, the method of treatment may for example comprise administration, such as oral administration, of:

A) in a dose of 100-600 mmol/day, such as 150-450 mmol/day, such as 170-350 mmol/day;
B) in a dose of 12-100 mmol/day, such as 16-75 mmol/day, such as 20-50 mmol/day; and/or
D) in a dose of 3-20 mmol/day, such as 4-15 mmol/day, such as 5-12 mmol/day.

Further, C) may be administrated in a dose of 12-100 mmol/day, such as 16-75 mmol/day, such as 20-50 mmol/day

The daily dose may be reached by administrating one or more doses per day to the patient. In one embodiment, the number of daily doses is one, two or three. For example, the patient may consume a drink formed from the composition in powderous form one, two or three times per day. Each dose or drink preferably comprises no more than 4.78 mmol/kg of A).

The method of treatment may for example be carried out for a period of at least one week.

Preferably, the first administration of the composition of the first aspect is carried out within 48 hours, such as within 24 hours, of the diagnosis of a coronavirus infection, such as a SARS-CoV-2 infection. Such a diagnosis may be RT-PCR-based.

For the patient/consumer, it is not necessary to take the substances of the present disclosure simultaneously. A therapeutic effect can also be achieved if the substances are taken separately or sequentially, preferably within a day and more preferably within an hour.

Accordingly, as a third aspect of the present disclosure, there is provided substances comprising

A) serine, glycine, betaine, N-acetylglycine, N-acetylserine, dimethylglycine, sarcosine and/or phosphoserine,
B) N-acetyl cysteine, cysteine and/or cystine,
C) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine and
D) nicotinamide riboside, quinolinate, deamino-NAD+, nicotinate D-ribonucleotide, nicotinamide D-ribonucleotide, nicotinate D-ribonucleoside, nicotinamide and/or nicotinate for simultaneous, separate or sequential use in a therapeutic method.

The therapeutic method is:

a method of treatment of a subject suffering from a severe acute respiratory syndrome (SARS); or
a method of treatment of a subject that is or has been infected with a coronavirus, typically a severe acute respiratory syndrome-related coronavirus.

The embodiments and examples of the first aspect apply to the second aspect mutatis mutandis.

A method of treating a subject, comprising administration to the subject of:

A) serine, glycine, betaine, N-acetylglycine, N-acetylserine, dimethylglycine, sarcosine and/or phosphoserine;
B) N-acetyl cysteine, cysteine and/or cystine;
C) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine; and
D) nicotinamide riboside, quinolinate, deamino-NAD+, nicotinate D-ribonucleotide, nicotinamide D-ribonucleotide, nicotinate D-ribonucleoside, nicotinamide and/or nicotinate.

The subject of the method is a subject suffering from a SARS or a subject that is or has been infected with a coronavirus.

The embodiments and examples of the first and second aspect apply to the third aspect mutatis mutandis.

The subject of the first, second and third aspect is preferably a human subject.

### EXAMPLE: CLINICAL TRIAL

An open label, randomized, controlled, investigator-initiated, multi-centre trial was carried out. The trial aimed to establish metabolic improvements in COVID-19 subjects by dietary supplementation with substances N-acetylcysteine, L-carnitine tartrate, nicotinamide riboside and serine plus standard therapy.

A mixture of the four substances was produced in Turkey according to GMP regulations by Pharmactive Company, Istanbul (www.pharmactive.com.tr/en/anasayfa.html) according to EU standards and packed according to GMP rules with the appropriate dosage. The mixture was produced as a soluble powder packed in 60 mL HDPE plastic bottles with screw caps. In addition to the four substances, the mixture contained strawberry aroma.

In the trial, the mixture was dissolved by patients in 200 mL preferably cold water before use.

Stability tests performed by RISE in Sweden show that the mixture is stable at 25° C. for at least twelve months in the plastic bottles.

Placebo products were produced at Pharmactive company using identical HDPE plastic bottles. Placebo products contained to be dissolved in 200 mL water.

### Study Population

The scope of the trial is 400 volunteering patients (men and women) aged 18 years or older, who have been diagnosed with COVID-19, meet all the inclusion criteria and do not meet any of the exclusion criteria. In a first stage of the trial, 100 patients were randomized and recruited. Provided that the statistical evaluation of the data obtained from the 100 patients are found to be satisfactory, a second stage of the study including an additional 300 patients will be carried out.

Inclusion Criteria:

Over 18 years of age;

Written informed consent obtained prior to any procedures related to the study;

Understanding all procedures applied within the scope of the study protocol;

Ambulatory patients with symptoms that have been diagnosed with RT-PCR test positivity and Chest tomography (CT) result positivity in the last 72 hours; and

Patients with stable clinical course and who could be treated on an ambulatory basis.

Exclusion Criteria:

Partial oxygen saturation below 93% and requiring immediate hospitalisation after diagnosis;

Hospitalised at the intensive care-unit upon initial examination;

Inability or unwillingness to give written informed consent;

Decision by physician that trial involvement will not be in the patient's best interest, or any condition that does not allow the protocol to be followed safely;

Patient considered as inappropriate for this study for any reason;

Active participation in another clinical study;

Uncontrolled Type 1 or type 2 diabetes;

Severe liver disease (e.g. Child Pugh score≥C, AST>5 times upper limit);

Known severe renal impairment (estimated glomerular filtration rate ≤30 mL/min/1.73 m2) or undergoing continuous renal replacement therapy, hemodialysis, peritoneal dialysis;

Significant cardiovascular co-morbidity (i.e. heart failure);

Suffering from phenylketonuria (contraindicated for NAC);

Known allergy for substances used in the study;

Pregnant or breastfeeding, or positive pregnancy test in a pre-dose examination; and

Receipt of any experimental treatment for COVID-19 within the 30 days prior to the time of the screening evaluation.

### Randomization and Blinding

The study subjects were randomized on a 3:1 basis. The study was open-labeled. A web-based randomization system was used to assign a randomization code for each patient. The investigator (or another responsible person at the investigational site) entered the web-based randomization system specific to the study through assigned username and password. After entering patient-related information (patient number, date of birth, patient initials), the system provided a randomization code for the future use.

### Dose and Administration

During the first stage of the trail, 25 patients were assigned to a group given hydroxychloroquine+placebo (placebo group) and 75 patients were assigned to a group given hydroxychloroquine+the mixture of the four substances (treated group).

After signing the informed consent forms, the first treatment (initial loading dose) was administrated by the responsible investigator based on the randomisation assignment. The remaining treatments were administered by the patient at home. Hydroxychloroquine treatment was administered at an initial dose of 2×400 mg (oral) followed by 400 mg/day (2×200 mg oral) for a total of 5 days.

The patients of the treated group were treated for two weeks by administration of the mixture of the substances in the doses shown table 1 below (twice/day; one dose in the morning, one dose after dinner):

TABLE 1

| | Dose | | | | Total |
|---|---|---|---|---|---|
| | per | | Total | | daily |
| | appli- | | daily | | molar |
| Substance | cation | | dose | | dose |
| Serine | 12.35 | g | 24.70 | g | 0.234 |
| L-Carnitine tartrate * | 3.73 | g | 7.46 | g | 0.031 |
| N-Acetylcysteine | 2.55 | g | 5.1 | g | 0.031 |
| Nicotinamide riboside | 1.0 | g | 2.0 | g | 0.0078 |

* 7.46 g/day L-carnitine tartrate (salt form of L-carnitine) was used to achieve 5.1 g/day of L-carnitine as active ingredient

Consequently, the total treatment period was 5 days for hydroxychloroquine and 14 days for the mixture of the four substances.

### Results

Patients that had been diagnosed with COVID-19 (by RT-PCR test) was contacted by telephone within 24 hours of the diagnostic test and asked if they wanted to participate in the clinical trial. Those who volunteered to do so visited the hospital shortly after the call (normally the same day) for a baseline examination. During this “Day 0” visit, the patients were also randomly assigned to a group (treated group or placebo group) as described above. The baseline demographics of the population that completed the first stage of the clinical trial are shown in table 2 below (7 out of the 100 recruited patients dropped out).

TABLE 2

| | Treated group | Placebo group |
|---|---|---|
| | (n = 71) | (n = 22) |
| Age | 35.0 (19.0 - 66.0) | 32.5 (20.0 - 58.0) |
| Sex | | |
| Male | 31 (44%) | 6 (27%) |
| Female | 40 (56%) | 16 (73%) |
| Body-mass index | 24.9 (16.8 - 37.8) | 241 (20.2 - 33.9) |
| (kg/m²) | | |
| Smoker | | |
| Yes | 17 (24%) | 6 (27%) |
| No | 54 (76) | 16 (73%) |
| Alcohol | | |
| Yes | 3 ( 4.2%) | 2 ( 9.1%) |
| No | 68 (95.8%) | 20 (90.9%) |
| Drug use | | |
| Yes | 16 (22.5%) | 4 (18.2%) |
| No | 55 (77.5%) | 18 (81.8%) |
| Symptoms and signs | | |
| Cough | 18 (25.4%) | 11 (50.0%) |
| Shortness of breath | 4 ( 5.6%) | 2 ( 9.1%) |
| (breath issue) | | |
| Malaise (tiredness) | 35 (49.3%) | 13 (59.1%) |
| Myalgia (muscle/joint pain) | 42 (59.2%) | 14 (63.6%) |
| Headache | 33 (46.5%) | 12 (54.5%) |
| Anosmia (smell and taste) | 19 (26.8%) | 6 (27.3%) |
| Sore throat | 19 (26.8%) | 3 (13.6%) |
| Nausea or vomiting | 5 ( 7.0%) | 0 |
| Diarrhea | 2 ( 2.8%) | 0 |

On Days 1-13 after the first visit (Day 0), the patients were called and asked to assess their symptoms and signs. On Day 14, the patients revisited the hospital for a final examination.

The present disclosure can present results from the first stage of the clinical trial. On average, the patients of the treated group were asymptomatic after 6.6 days. In contrast, the patients of the placebo group were on average asymptomatic after 9.3 days.

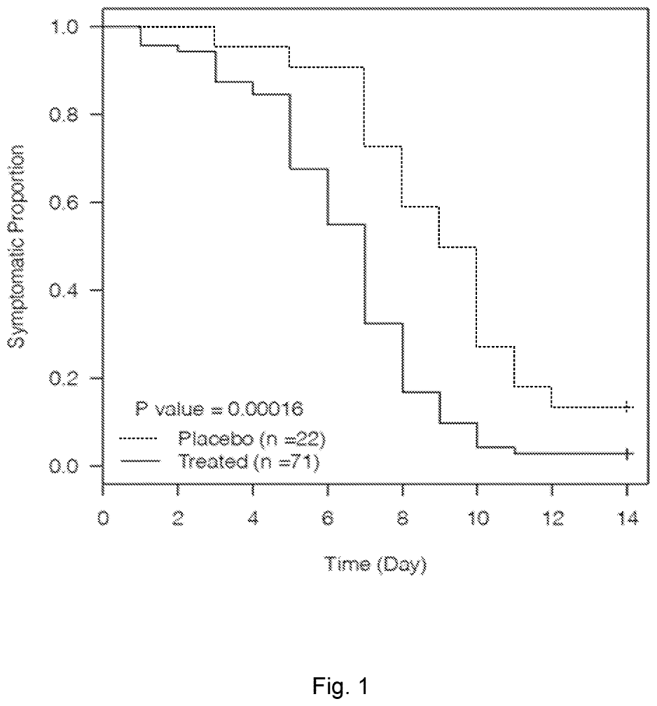

As can be seen in the Kaplan-Meier graph of FIG. 1, the effect of the treatment was significant (p=0.00016). Further, FIG. 1 shows that almost all the patients in the treated group were asymptomatic on the last day (Day 14), whereas about 14% of the patients in the placebo group still had symptoms at that time.

In a multivariate analysis, after adjusting for covariates, including age, gender, drug, cigarette, and alcohol usage, the treatment remained significantly associated with time to recovery (p<0.0003, hazard ratio (HR)=2.68, 95% CI: 1.57-4.59).

1. A method of treating a human subject that is or has been infected with a coronavirus, comprising administration to the subject of:
A) serine, glycine, betaine, N-acetylglycine, N-acetylserine, dimethylglycine, sarcosine and/or phosphoserine;
B) N-acetyl cysteine, cysteine and/or cystine;
C) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine; and
D) nicotinamide riboside, quinolinate, deamino-NAD-+, nicotinate D-ribonucleotide, nicotinamide D-ribonucleotide, nicotinate D-ribonucleoside, nicotinamide and/or nicotinate;
thereby treating the human subject;
wherein the subject does not have non-alcoholic fatty liver disease.

2. The method of claim 1, wherein said coronavirus is a severe acute respiratory syndrome-related coronavirus.

3. The method of claim 2, wherein said severe acute respiratory syndrome-related coronavirus is severe acute respiratory syndrome coronavirus 2 (SARS-CoV-2).

4. A method of treating a human subject suffering from a severe acute respiratory syndrome (SARS), comprising administration to the subject of:
A) serine, glycine, betaine, N-acetylglycine, N-acetylserine, dimethylglycine, sarcosine and/or phosphoserine;
B) N-acetyl cysteine, cysteine and/or cystine;
C) optionally carnitine, deoxycarnitine, gamma-butyrobetaine, 4-trimethylaminoniobutanal, 3-hydroxy-N6,N6,N6-trimethyl-L-lysine, N6,N6,N6-trimethyl-L-lysine and/or lysine; and
D) nicotinamide riboside, quinolinate, deamino-NAD+, nicotinate D-ribonucleotide, nicotinamide D-ribonucleotide, nicotinate D-ribonucleoside, nicotinamide and/or nicotinate;
thereby treating the SARS;
wherein the subject does not have non-alcoholic fatty liver disease.

5. The method of claim 4, wherein said SARS is COVID-19.

6. The method of claim 1, wherein the administration of A), B), optionally C) and D) is simultaneous, separate or sequential.

7. The method of claim 1, wherein A), B), optionally C) and D) are contained in a composition that is administered to the subject.

8. The method of claim 7, wherein the composition is a powderous mixture.

9. The method of claim 1, wherein the molar ratio of A) to B) is between 20:1 and 1:4.

10. The method of claim 9, wherein the molar ratio of A) to B) is between 16:1 and 1.5:1.

11. The method of claim 1, wherein the molar ratio of A) to C) is between 150:1 and 1:1.

12. The method of claim 1, wherein the molar ratio of A) to D) is between 250:1 and 1.5:1.

13. The method of claim 12, wherein the molar ratio of A) to D) is between 150:1 and 3:1.

14. The method of claim 1, wherein A) is serine.

15. The method of claim 1, wherein D) is nicotinamide riboside.

16. The method of claim 1, wherein said therapeutic method comprises oral administration of the substances.

17. The method of claim 1, wherein said therapeutic method further comprises administration of an anti-viral drug, chloroquine or hydroxychloroquine.

18. The method of claim 1, wherein said administration of the anti-viral drug, chloroquine or hydroxychloroquine is carried out prior to or concurrently with said administration of A), B), optionally C) and D).

19. The method of claim 1, wherein said therapeutic method comprises administration of:
A) in a dose of 0.48-24 mmol/kg/day;
B) in a dose of 0.31-3.05 mmol/kg/day;
optionally C) in a dose of 0.100-2.50 mmol/kg/day; and
D) in a dose of 0.039-0.39 mmol/kg/day.

20. The method of claim 1, wherein said therapeutic method comprises administration of:
A) in a dose of 100-600 mmol/day;
B) in a dose of 12-100 mmol/day;
optionally C) in a dose of 12-100 mmol/day; and
D) in a dose of 3-20 mmol/day.
